(19) United States
(12) Patent Application Publication
(10) Pub. No.: US 2025/0339823 A1
(43) Pub. Date: Nov. 6, 2025

(54) METHODS, DEVICES, SYSTEMS AND PRODUCTS FOR PREPARING COMPOSITIONS FOR CARE AND REPAIR OF VARICOSE VEINS

(71) Applicant: VASCULAR BARCELONA DEVICES, S.L., Barcelona (ES)

(72) Inventors: Enrique ROCHE REBOLLO, Barcelona (ES); Guiu LLUSA MELENDEZ, Barcelona (ES); Federico GREGO MAYOR, Barcelona (ES)

(21) Appl. No.: 19/273,393

(22) Filed: Jul. 18, 2025

Related U.S. Application Data
(63) Continuation of application No. PCT/EP2024/051135, filed on Jan. 18, 2024.

(30) Foreign Application Priority Data
Jan. 18, 2023 (EP) 23382040.6

(51) Int. Cl. B01F 23/235; A61J 1/20; A61K 9/12; B01F 23/20; B01F 23/23; B01F 23/237; B01F 33/453; B01F 35/00; B01F 35/71; B01F 35/75; B01F 101/22; B65B 5/04; B65B 31/04; B65D 77/20; B65D 81/20

(52) U.S. Cl. CPC B01F 23/2351; A61J 1/2096; B01F 23/2368; B01F 23/237612; B01F 23/23762; B01F 23/291; B01F 33/4535; B01F 35/187; B01F 35/7174; B01F 35/75425; B65B 5/04; B65B 31/046; B65D 77/2024; B65D 81/2076; A61K 9/122; B01F 2101/22

(57) ABSTRACT

Containers for the production of a foamed sclerosant composition are provided. The containers includes a container body for foaming having one or more sidewalls extending between a top and a bottom of the container body, a foaming space being defined in an interior of the container body. Further, the foaming space contains a mixing component configured for rotating, particularly on the bottom of the container body, and including a magnetic part and a shape suitable for mixing. The container also includes a female coupling member for mating with a tip of a syringe, such that the syringe can be used for extracting the foamed sclerosant composition, and the container includes at least one opening which is covered by a gas-permeable barrier.

### CROSS-REFERENCE TO RELATED APPLICATIONS

The present application relates to and claims the benefit and priority to International Application. No. PCT/EP2024/051135, filed Jan. 18, 2024, which claims the benefit and priority to European Patent Application No. EP23382040.6 filed on Jan. 18, 2023, each of which is incorporated herein by reference in its entirety.

### FIELD

The present disclosure is related to the technical field of vascular medicine, and more particularly to the field of treatments for varicose veins and other vascular problems such as, for example, spider veins and/or hemorrhoids. Specifically, the present disclosure relates to devices, systems, products and methods for obtaining a foam, which can be used for the treatment of the affected veins. The present disclosure further relates to the composition of the foam itself obtained by any of the procedures described throughout the present disclosure, as well as the use of the devices, systems and products in order to treat varicose veins and other vascular problems such as e.g. spider veins and/or hemorrhoids. Furthermore, the present disclosure also relates to methods for the manufacture of such products to treat varicose veins and other vascular problems.

### BACKGROUND

Varicose veins occur when the venous valves (which prevent the backflow of blood) do not work properly. As a result, the vein walls are weakened, and they can become deformed and dilated. Due to the fact that the valves do not work properly, the blood may recirculate and short-circuits may be created. Subsequently, the veins may become progressively dilated. As a result, the varicose veins can become more visible, and can be full of bends, and become more voluminous. The evolution of this pathology may lead to consequences beyond mere cosmetic ones, such as discoloration of the skin, pain, and swelling of the extremities due to the effect of venous hypertension.

According to the Spanish Society of Angiology and Vascular Surgery (SEACV) in Spain, varicose veins affect from 30 to 33% of the adult population in industrialized countries.

The veins most commonly affected are those located in the legs, although varicose veins may occur interiorly as well: e.g. varicose veins in the esophagus, around organs located in the pelvis (pelvic and ovarian varicose veins) or at or near the most distal part of the digestive tube, near the anus (hemorrhoids).

Nowadays, there are many different treatments and/or strategies to mitigate or eliminate these problems.

A common technique to treat varicose veins is sclerotherapy. This technique is less aggressive and less painful than other techniques such as endovenous techniques or radiofrequency therapies. Further, sclerotherapy needs no anesthesia.

Sclerotherapy involves the injection of a liquid (or foam when shaken) with the ability to irritate the vascular endothelium, which is a thin layer or lining inside the vein that is in contact with the bloodstream. The products internationally approved for this use are lauromacrogol (also known named as polidocanol and commercially available as etoxiesclerol®), and sodium tetradecyl sulfate.

The advantage of using such a product as a foam is based on the enhancement of its effect due to the larger contact surface with the endothelial wall. The larger contact surface offers the possibility of dose reduction. Also, the visibility of the drug as a foam using ultrasounds through the ultrasound scanner is improved (Schadeck M, Allaert FA. Duplex scanning in the mechanism of the sclerotherapy: Importance of the spasm. Phlebology 1995; Suppl1: 574-576).

The effect produced by the foam on the endothelium involves the injury of the cell layer of the vein, whereby thrombosis of its content is produced. Later, this vein suffers a fibrosis process (retraction and disposal) and the vein eventually may disappear after several months. This process can be faster or slower depending on the size of the vein or the potency of the varicose agent. Therefore, it is sometimes necessary to apply several sessions on the vein.

In recent years, this procedure has been developed a lot further and there has been a growing interest from the scientific community to obtain methods suitable for the manufacture of such foams in a safe and convenient way.

Although there are several methods to eliminate or remove varicose veins, the less aggressive and less disabling treatment so far, and the one which can be used for a wide variety of pathologies is the ultrasound-guided Foam Sclerotherapy using polidocanol or other sclerosing agents. Sclerotherapy is the least invasive treatment of varicose veins known to date as it can be performed in a physician's office and in a completely ambulatory way. Therefore, the present disclosure is focused on the use of this technique.

In 1995, Dr. Juan Cabrera presented the results of the application of a foam that he had developed with his son, the pharmacist Juan Cabrera (Cabrera J. et al. “Treatment of Varicose Long Saphenous Veins of Sclerosants in Microfoam Form With: Long-term Outcomes”. Phlebology (2000) 15; 19-23). This foam was characterized by its density and high solubility thanks to the use of a mixture of physiological gases.

Furthermore, he managed to achieve a type of foam with a very small and uniform bubble size, thanks to his method of using a mixer. By using a mixture of physiological gases, the foam had greater security and stability. This foam was called “microfoam” due to the small bubble size, uniformity and stability.

Shortly after, the maximum popularization of the sclerosing foam came from Lorenzo Tessari (Tessari L., Cavezzi A., Frullini A., Preliminary experience with a new sclerosing foam in the treatment of varicose veins. Dermatol Surg 2001 January; 27 (1): 58-60) who published his experience using a foam easily manufactured through a procedure called “The Tessari Method”. The method consists of agitating a sclerosing liquid using two syringes connected via a three-way tap. By means of successive alternating movements with each of the syringes connected to the gas/liquid mixture, a mix of foam, located at the inner part of the syringe, was achieved. However, this manufacturing technique, in spite of being the most widespread is not the most effective, since a relatively unstable and heterogeneous foam is obtained.

In recent years numerous articles and papers have been published about safety profiles, side effects, and potential complications arising from the use of these products. Thus, it appears demonstrated that the best foam to be used is one with a mixture of O₂/CO₂ at different concentrations. With this arrangement, the solubility and diffusion in the blood are very high as opposed to atmospheric gas foams. Additionally, the foam stability is linked to the size of the bubble. Also, it has been found that the foam is more stable when the bubble diameter is more homogeneous.

As has already been mentioned, although there are a lot of manufacturing systems of sclerosing foam, the most common foam (and the one which is normally used around the world) is the foam obtained with the Tessari method. However, this method has many problems of standardization and homogenization. This system consists of mixing air flow with the selected liquid, either polidocanol or sodium tetradecyl sulfate (commercially known as sotradecol®). This foam can have medium size bubbles, but of irregular size and the foam becomes unstable after a few seconds of its formation. Additionally, the use of atmospheric gas as a vehicle for the sclerosing foam limits the foam administrable per session.

US2017144115 discloses a container for the production of a foamed sclerosant composition, kits and systems including such a container, and methods for preparing a foamed sclerosant composition using such containers. In an aspect, the container comprises a container body and a mixing element disposed in the container body, such that a foaming space is defined in an interior of the container body between the sidewalls and the mixing element. The mixing element may be configured to be operatively coupled with a rotating actuator without the actuator reaching the foaming space. In particular, the mixing element may have a magnetic core, such that the mixing element can be set in motion when placed on a magnetic stirrer.

WO2017085209 relates to a container for the production of a foamed sclerosant composition, to kits and systems including such a container, to methods for preparing a foamed sclerosant composition using such containers, and to foamed sclerosant compositions obtainable by such methods. In an aspect, the container comprises a sealed sterile container body having one or more sidewalls extending between a top and a bottom of the container body and defining a foaming space. The container further comprises a mixing element disposed in the container body. The container contains a previously introduced gas and liquid sclerosant composition. The mixing element is configured to be operatively coupled with a rotatable actuator without the actuator reaching the foaming space. A medical professional may select the appropriate quantity and concentration for every treatment.

WO2020038928 also relates to a container for the production of a foamed sclerosant composition, to kits and systems including such a container, to methods for preparing a foamed sclerosant composition using such containers, and to foamed sclerosant compositions obtainable by such methods. In an aspect, the container comprises a pressure equalizer for equalizing pressure inside and outside the foaming space.

These documents disclose methods and devices that facilitate quick production of good quality sclerosant foam with relatively simple tools and in a highly sterile manner.

However, there is still a need for devices, products, systems and methods that further improve the production of sclerosant foam.

### SUMMARY

In a first aspect, the present disclosure provides a container for the production of a foamed sclerosant composition. The container comprises a container body for foaming comprising one or more sidewalls extending between a top and a bottom of the container body. Thus, the container body defines a foaming space in an interior of the container body. Further, the foaming space contains a mixing component configured for rotating, and optionally for rotating on the bottom of the container body. In addition, said mixing component comprises a magnetic part and a shape suitable for mixing. The mixing component is also configured to be operatively coupled with a magnetic stirrer. Further, the container comprises a coupling member for mating with a tip of a syringe. The coupling member may be located at or near the bottom surface of the container body such that the syringe can be used for extracting the foamed sclerosant composition. Moreover, the container comprises at least one opening which is covered by a gas-permeable barrier.

In accordance with this aspect, a container is provided which is specifically suited for the preparation of a sclerosant foam in which a syringe may be used for the introduction of a liquid sclerosant composition. The introduction of a liquid sclerosant composition can produce an instant overpressure inside the foaming space, i.e. the (air) pressure inside the foaming space may be higher than the ambient pressure, but this overpressure is quickly equalized due to the presence of the gas-permeable barrier in fluid communication with atmospheric air, without the need for a user action or activation. Having a substantially similar pressure inside and outside of the container reduces the formation of gas bubbles in the syringe during foaming aspiration. Thus, the gas-permeable barrier may improve foam quality, and more specifically foam quality during and after foaming aspiration. At the same time, the barrier may block particles and contaminants from entering into the foaming space.

Similarly, after extraction of foam, the pressure inside the container may be reduced, and thereby some ambient air may be sucked into the container. The gas-permeable barrier allows the gas flow to happen but avoids contamination of (micro)particles inside the container.

Providing a gas-permeable barrier also provides advantages when the container is to be filled with a specific composition of gas, other than air, as will be explained hereinafter.

The same or a different syringe than the one used for the introduction of the sclerosant composition may be used for aspiration or extraction of foam from the foaming space. Then, the same syringe may be used for injection into a patient's vein.

In examples, the container may be substantially sterile, e.g. the container body may be sterile or the foaming space inside the container body may be sterile.

In some examples, the gas-permeable barrier may have a relatively high capacity to transfer gas across the same. The permeability of the gas-permeable barrier may be estimated by applying the Bendtsen method wherein a sample of 10 cm² of the barrier material is subjected to a pressure difference of 1.47 kPa, and the volume of air through the sample is measured. Thus, in some examples, the gas-permeable barrier may provide a permeability of between 50-400 ml/minute under the above-mentioned conditions, and more specifically between 150-250 ml/minute.

Other parameters indicative of the degree of air transfer across the barrier are air permeability and air permeability coefficient. These parameters are related to the porosity, the shape of the pores in the barrier, and their level of connectedness among others. Thus, a barrier with high air permeability offers little resistance to air transfer across the same.

In some examples, the gas-permeable barrier may comprise a non-woven fabric. Further, in examples, the gas-permeable barrier may comprise polypropylene, polyethylene or a combination of the same. These materials may be integrated into one layer or a plurality of layers forming the barrier.

In some examples, in addition to a gas-permeable barrier, a further fluid tight closure may be provided over the gas-permeable barrier. An orifice may be covered by the gas-permeable barrier. The same orifice may be covered by a releasable cover such as an aluminum foil sealed over the orifice. The cover may have a shape adapted for easily pulling off the cover and may include a handle or grip which a user may grip for handling.

In a specific example, a gas-permeable barrier may be provided at an inside of the container to close the orifice. At the outside of the container, the fluid tight closure may be provided to cover the same orifice.

In some examples, the coupling member of the container body may be a female coupling member. The female coupling member of the container body may comprise a Luer taper. A female coupling member as used throughout the present disclosure may be regarded as a coupling member with a suitable receptacle or opening for receiving a male coupling member, and in the present case the male coupling member of a distal end of a syringe. The female coupling member may have the shape of a socket or a sleeve.

The Luer taper is a standardized system of small-scale fluid fittings used for making leak-free connections between a male-taper fitting and its mating female part on medical and laboratory instruments, including e.g. syringe tips.

The Luer taper of the female coupling member may be a female Luer slip. Alternatively, a Luer-lock may be used. Luer lock fittings are securely joined by means of a tabbed hub on the female fitting which screws into threads in a sleeve on the male fitting. Slip tip (Luer-slip) fittings simply conform to Luer taper dimensions and are pressed together and held by friction (they have no threads).

In some examples, the female coupling member for mating with a syringe may further comprise a one-way valve for the introduction of a liquid sclerosant composition. The one-way valve may ensure that the introduction of a liquid sclerosant composition can be done without compromising the environment in the foaming space, e.g. a sterile environment. The one-way valve may be a simple sheet or foil which is cantilever mounted to allow pivoting and thereby provide a one-way access to the foaming space. In some examples, the one-way valve may be arranged downstream from the point of injection of the liquid composition.

In some examples, the female coupling member may comprise a barrier. The barrier may be removable, releasable, or breakable. Further, the barrier may be a gas-impermeable barrier or a gas-permeable barrier with the same or substantially similar characteristics as previously discussed for the gas-permeable barrier of the opening of the container.

In some examples, the mixing component configured to be operatively coupled with a magnetic stirrer may be a disc comprising a magnetic part and a shape suitable for mixing. Such a mixing component can be activated and controlled by providing a changing magnetic field. In particular, the magnetic stirrer may be configured to cause a rotating magnetic field.

In some examples, the container may comprise a physiological gas or a mixture of physiological gases, and optionally may be a mixture of O₂ and CO₂. In some examples, the gas inside the container may comprise more than 95% of physiological gases, and more specifically more than 98% of physiological gases. In examples, the gas inside the container may be nearly 100% physiological gas(es). Further, in some examples, the mixture of physiological gases may have a percentage of O₂ between 70%-50% and a percentage of CO₂ between 30%-50%. The foam prepared with physiological gases may be suitable for the treatment of certain patients and/or veins which cannot be treated by foam prepared with (sterile) air.

In another aspect, the present disclosure provides a product to produce a foamed sclerosant composition. The product comprises a container according to the present disclosure and filled with a gas. In the product, the container is provided in a sealed package. The package defines an interior space filled with the same gas, and configured to house the container. Further, the interior space of the sterile package and the foaming space of the container may be in fluid communication.

According to this aspect, the container may remain isolated from atmospheric air during transportation and storage, and thereby retain the intended gas composition inside the container. In examples, during storage and transportation, the interior space of the package and the foaming space may be in communication with each other e.g. through the gas-permeable barrier. Thus, a user may benefit from the ease of use of the container, whereas at the same time the gas composition inside the container may remain substantially the same for a prolonged period of time. In some examples, the product may be substantially sterile, i.e. an interior of the container may be sterile.

In examples, the gas inside the package may have a composition substantially similar to the gas composition of the container, particularly a physiological gas or mixture of physiological gases. In a specific example, the container may be filled with 65% O₂ and 35% CO₂. The same composition may be used in the packaging. Even if gas exchange from inside the container to outside the container occurs, the same gas composition may be maintained inside the container.

Gas exchange can occur through the gas-permeable barrier of the container in some examples, but it will not modify the composition of the gas inside the container, and thus will not affect the composition of the foamed sclerosant composition. It was further surprisingly found that the material of the container (e.g. PET) may leak at a very low rate, e.g. 2% of original volume in the container per month. Even such a slow leaking rate may lead to a different than intended foam composition. To avoid such problems when the composition of the gas and foam is critical e.g. in terms of O₂, CO₂ or N₂, the packaging may be filled with gas of substantially the same composition. So even in embodiments wherein all openings of the container are sealed, and no fluid communication is supposed to be possible between the foaming space and the packaging, providing the specific gas composition in the packaging can still be useful.

In some examples, the sealed package comprises a lid configured to be peeled off. Such a lid enables ease of manufacturing and ease of use. The peelable lid may be heat sealed to the package.

In examples, the sealed sterile package comprises a layer that is impermeable to gas. Thus, the sterile package may remain with substantially the same gas composition during a prolonged period of time, e.g. months or even years.

In an additional aspect, a kit comprising the product previously disclosed and a pre-loaded syringe comprising the sclerosant composition is provided.

In a further aspect, a method for manufacturing a product to produce a foamed sclerosant composition is provided. The method comprises providing a package defining an interior space for housing a container, and providing a container as previously disclosed. The container is provided inside the package, and the interior space of the package and the foaming space of the container are in fluid communication through the gas-permeable barrier. Further, the method comprises filling the package and the container with a physiological gas or mixture of physiological gases. In addition, the method comprises sealing the package.

In examples, the method comprises sterilizing the product. This may be performed in a variety of manners including using moist heat (steam), dry heat, radiation, ethylene oxide gas, vaporized hydrogen peroxide, and other sterilization methods. The step of sterilizing the product may be performed before or after sealing the package. Depending on this, a sterilization technique may be more suitable than others, e.g. radiation may be used for sterilization after sealing the package.

In some examples, the method for manufacturing a product comprises reducing an air pressure around the package (and container) below atmospheric pressure before filling the package (and container) with a physiological gas or mixture of physiological gases. Further, reducing the air pressure may bring the air pressure around the package below 0.1 bar, and more specifically below 0.03 bar.

In examples, the mixture of physiological gases used to fill the package and the container may be substantially the same as previously discussed for the product.

In some cases, after filling of the container and packaging with gas, the container may be closed. In particular, a stopper may close off the female coupling member and a fluid tight seal may be arranged over the gas-permeable barrier.

In yet another aspect, a system is provided which comprises a container as previously disclosed and an actuator configured to be operatively coupled to the mixing component configured for rotating. The actuator may in particular be a magnetic stirrer.

In a further aspect, a method for preparing and extracting a foamed sclerosant composition is provided. The method comprises providing a container to produce a foamed sclerosant composition having a sterile container body, as hereinbefore explained. The method also comprises providing a syringe filled with a liquid sclerosant composition. Additionally, the method comprises mating the syringe with the coupling and injecting the liquid sclerosant composition into the foaming space, operating the actuator to rotate the mixing component configured for rotating on the bottom of the sterile container body and create a foamed sclerosant composition, and extracting the obtained foamed sclerosant composition.

According to this aspect, a substantially sterile and relatively easy method for preparing and extracting a foamed sclerosant composition is provided allowing the coupling of a syringe with the container having the foaming space.

In examples, the mixture of physiological gases inside the foaming space may be substantially the same as previously discussed for the product.

In some examples of the method, providing the container may comprise providing a sealed sterile package defining an interior space comprising the container. Further, the method may comprise opening the sealed sterile package to access the container and mating the syringe with the coupling in less than 15 minutes from the opening of the sealed sterile package, and more specifically in less than 5 minutes from the opening.

In yet a further aspect, a foamed sclerosant composition obtainable by such a method is provided.

In some examples, the syringe used for the introduction of the liquid sclerosant composition remains mated with the female coupling member while the foam is being created. If the syringe remains coupled with the container, the chances of contaminating the foam are reduced. After the foam has been created, the same syringe may be used for aspiration or extraction of the foam. The gas-permeable barrier may also be useful to again equalize the pressures so that a good quality foam may be extracted.

In some examples, an actuator may be operated at a first speed of rotation to create a foamed sclerosant composition, and the actuator may be operated at a second speed of rotation while the foamed sclerosant composition is being extracted, wherein the first speed of rotation is different from the second speed of rotation. In particular, a first high speed of rotation may be used for rapid preparation of foam of good quality (e.g. small bubbles), and the rotation continues as the foam is being extracted. The continued rotation forces the created foam to the outer area of the foaming space, from where it can be aspirated.

In some examples, the foamed sclerosant composition obtainable by any of the methods, systems and products described herein may be for use in the treatment of varicose veins, spider veins or hemorrhoids.

### BRIEF DESCRIPTION OF THE DRAWINGS

Particular implementations of the present disclosure will be described in the following by way of non-limiting examples, with reference to the appended drawings, in which:

FIG. 1 schematically illustrates an example of a container for producing a foamed sclerosant composition;

FIG. 2 schematically illustrates an example of a product for producing a foamed sclerosant composition; and

FIG. 3 schematically illustrates a block diagram of an example of a method for manufacturing a product according to the present disclosure.

FIG. 4A-4F schematically illustrates a sequence of steps in an example of a method for the production of a foamed sclerosant composition.

FIGS. 5A and 5B schematically illustrate another example of a container for producing a foamed sclerosant composition.

### DETAILED DESCRIPTION

The expression “therapeutically effective amount” as used herein, refers to the amount of the foamed composition that, when administered, is sufficient to treat the diseases to which it is addressed. The specific dose which depends on both the volume and the drug concentration of the foamed sclerosant composition to obtain a therapeutic benefit may vary depending on the particular circumstances of each patient.

As previously mentioned, an aspect of the present disclosure relates to a foamed sclerosant composition obtainable by any of the methods herein described. The expression “obtainable by” is used herein for defining the foamed sclerosant composition by its preparation process. In particular, it refers to the foamed composition that can be obtained through the preparation process which comprises the previously commented steps. For the purposes of the present disclosure, the expressions “obtainable”, “obtained” and similar equivalent expressions are used interchangeably and, in any case, the expression “obtainable” encompasses the expression “obtained”. Throughout the present disclosure, the terms sclerosant and sclerosing are used interchangeably. Similarly, sclerosing foam, and foamed sclerosant composition are used interchangeably as well.

For the purposes of the present disclosure, the term foamed sclerosing composition refers to a composition of a foam capable of bringing about a sclerosing effect, i.e. a composition for use as a medicament for intravenous injection, which is capable of causing an injury to the vessel wall by endothelial vacuolation of the epithelial cell membrane (the layer in contact with the bloodstream). Thus, the foamed sclerosing composition irritates the inner surface of the vein just producing the formed thrombus formation by platelets and aggregates. Similarly, the term liquid sclerosing composition refers to a composition in liquid form including a sclerosing agent. The liquid sclerosing composition forms an ingredient to obtain the foamed sclerosing composition. The sclerosing compositions according to examples of the present disclosure comprise a sclerosing agent and also a suitable vehicle which can be injected without toxicity in the affected veins. In some examples, the liquid is selected from sterile water (particularly distilled water) and physiological saline. Examples of sclerosing agents that can be present in the sclerosing compositions of examples of the present disclosure include, without limitation, polidocanol, sodium tetradecyl sulfate, chromated glycerin, hypertonic saline, sodium morrhuate and sclerodex (hypertonic saline in combination with dextrose). In a particular example, the sclerosing composition used comprises polidocanol and water. In another embodiment, the sclerosing composition further comprises glycerin.

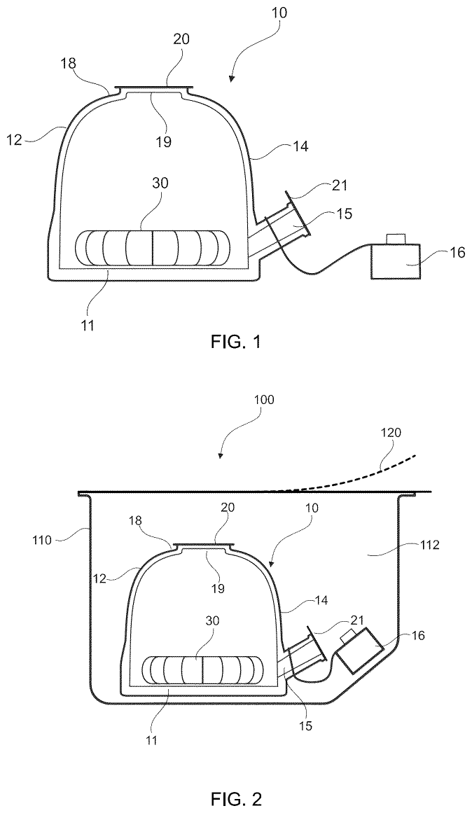

FIG. 1 schematically illustrates an example of a container for producing a foamed sclerosant composition. A container 10 according to this example may include a dome shaped container body 12 with a bottom wall 11. The bottom wall 11 may be separately manufactured or may be manufactured integrally with the dome shaped container body 12. The dome shaped container body 12 may have a top 18 with an orifice 19. In this example, the orifice 19 is covered by a gas-permeable barrier 20. Even though the orifice 19 is herein depicted to be at the top of the container body 12, but in other examples, the orifice 19 may be located elsewhere in order to allow exchange of gases through the barrier 20, but avoiding contamination of particles, aerosols, liquids, other contaminants etc.

The dome shaped body 12 is formed by a curved side wall 14. Thus, a foaming space is defined in the interior of the container 10. The container 10, both dome shaped body 12 and bottom 11, may be made from a suitable polymer such as Polyethylene terephthalate (PET) or other suitable materials such as plastics generally accepted in the manufacture of medical devices. Other materials such as different types of glass with different material compositions, or materials with crystalline microstructure may also be used to manufacture the dome shaped body 12.

In some examples, the material of which the container is made is transparent to allow visual inspection of the formation of the foam. The dome shaped container body 12 and the bottom part 11 may be made by injection molding within the same mold or by separate molds. Other manufacturing processes could also be used.

If manufactured separately, the bottom 11 and the dome shaped body 12 may be joined by ultrasonic welding or other appropriate ways for joining plastic components.

A gas may be introduced into the foaming space during manufacture and assembly. The gas may be air but may also be a mixture of physiological gases, e.g. a mixture of carbon dioxide (CO₂) and oxygen (O₂). In specific examples, the amount of air or nitrogen in the container may be less than 10%, or less than 5%, preferably less than 2% or less than 1% of the volume inside the container. The remaining amount may be a mixture of physiological gases, specifically oxygen and carbon dioxide.

More precisely, in specific examples, the mixture of physiological gases may comprise a percentage of O₂ between 70%-50% and a percentage of CO₂ between 30%-50%. The percentages of the gas composition generally are percentages by volume.

As provided, the foaming space also includes a mixing component 30 configured for rotating on the bottom 11 of the container body 10. The container 10 with previously introduced gas and mixing component 30 for rotating may thus be provided to a medical professional.

The container 10 may be sterilized prior to providing it to a medical professional or it may be sterilized on site by a medical professional. Thus, in some examples the container 10 according to this example may be sterilized and wrapped. For example, plastic foil or wrapper may be provided around the container as delivered to a medical professional.

In other examples, the container 10 may be housed in a package, as will be discussed in regard to FIG. 2.

As shown in FIG. 1, the container 10 according to this example further includes a female coupling member 15 in the form of a socket with a stopper or lid 16. The female coupling member 15 may comprise external threads and the stopper 16 may comprise mating threads for easy attachment and detachment of the stopper. Alternative joints for female coupling member 15 and stopper 16 may however also be foreseen, e.g. a press or snap fit.

In examples, the female coupling member 15 may comprise a Luer taper, and particularly a Luer slip. The Luer taper may be sized such that it can mate with a tip of a syringe, in particular a hypodermic syringe. The tip of the syringe may be pressed into the Luer taper to provide a leak free fitting. In some examples, the syringe may be rotated to lock the syringe in the female coupling member 15 by means of a Luer lock.

In this example, the female coupling member 15 is integrally formed with the container 10 and in particular is provided on the sidewall 14, in proximity to the bottom 11. In some examples, the center of the female coupling member 15 is on the sidewall 14, at a height of 20 mm or less, i.e. up to 20 mm away from the bottom 11 of the container 10. Specifically, the center of the female coupling member 15 may be on the sidewall 14 up to 10 mm away from the bottom 11 of the container 10. However, other suitable positions for the female coupling member 15 may be envisaged, particularly positions that allow extraction of the foam near the bottom of the foaming space. The stopper 16 may be made from polyethylene or other suitable materials.

Although not shown in this specific example, the female coupling member 15 or its connection to the foaming space may include a one-way valve, or any other element that facilitates passage from a liquid inside the syringe, and substantially impedes passage of gas from the inside to the outside.

In this example, the female coupling member 15 has an inclination with respect to a horizontal plane for easy insertion and extraction from the syringe.

An example of an assembly process of a container 10 is the following: after injection molding of the container 10, and before attachment of the bottom 11, the mixing component 30 is introduced into the foaming space of the container, and the housing is then closed. Sterile gas may be introduced through a suitable port, i.e. opening 19 and/or female coupling member 15 (e.g. air, and/or a mixture of physiological gases) forcing the evacuation of the gas previously present inside the container 10.

The gas-permeable barrier 20 of the container 10 may be a waterproof membrane, i.e. it may limit the ingress of liquid into the container 10. The waterproofing capacity of the membrane may be measured in terms of the height of the column of water that the membrane may hold before losing its permeability. In some examples, the waterproof membrane may have a waterproofing capacity of 1500 mm of column of water or more, and more specifically 5000 mm or more. Further, the gas-permeable barrier 20 may limit the penetration of aerosols and other suspension particles, which may contaminate the interior foaming space.

Additionally, the gas-permeable barrier 20 may have a relatively high capacity to transfer gas across the same.

In the present example, the gas-permeable barrier 20 comprises a polyethylene non-woven fabric, but other types of fabrics and materials may be used. For example, the barrier 20 may comprise polypropylene or other suitable materials.

The function of the gas-permeable barrier 20 is to balance pressure inside container 10 with pressure outside the container 10. Equilibration of pressures can avoid potential formation of undesirable large bubbles in the sclerosant foam.

As illustrated in FIG. 1, the female coupling member 15 may comprise a barrier 21, e.g. a gas-permeable barrier of the same of substantially similar characteristics to the gas-permeable barrier 20 covering the opening 19. Barriers with other mass transfer characteristics may also be used, e.g. a gas-impermeable barrier. The barrier 21 may protect the female coupling member 15 against the ingress of contaminants.

Barriers 20, 21 may be made of medical paper or may be made of Tyvek® commercially available from DuPont. Tyvek® is made of synthetic flashspun high-density polyethylene fibers. Tyvek® is waterproof but allows water vapor to penetrate. In one example, Tyvek^{R} Allover 2FS/ACT2100™ may be used as material for the barrier. This product corresponds to Tyvek^{R} 2FS coated with a heat sealable coating and is a known packaging material for medical products. The air permeability measured with the aforementioned Bendsten mention is around 200 ml/minute The material is also suitable for sterilization, e.g. with irradiation or with ethylene oxide.

The barrier may be attached to the container to cover the opening(s). The barrier may be adhesively attached, or through ultrasound welding, or heat sealing or any other suitable method. In examples, the barrier(s) may be arranged at a substantially flat outer surface. For example, the dome-shaped walls may comprise a portion at near the top that is substantially flat which facilitates attachment of a suitable barrier.

The opening 19 may have a relatively small diameter, sufficient for gas exchange through the gas-permeable barrier 20. Without limitation, the opening 19 can be 2.5 to 15 mm in diameter. Specifically, the opening 19 may be between 5 and 10 mm in diameter.

Further, the opening 19 may particularly be located where it is not exposed to the foam created, for example at a top 18 or high point of container 10. The opening 19 may also include a microbial barrier to reduce potential exposure of the foam to airborne particles and microbes upon equilibration of pressures and withdrawal of the foam. The microbial barrier and gas permeable barrier may be two separate elements or may be combined in a single barrier element.

In the present example, the barrier 19 is not manipulated during normal use. The barrier 21 may be manipulated. In order to close off the female coupling member with lid 16, the barrier 21 may either be pierced or removed.

The mixing component 30 configured for rotating on the bottom 11 of the container may comprise a magnetic element, in particular a magnetic core. The magnetic element may be provided in a receptacle, e.g. a centrally arranged receptacle. The magnetic element may be a permanent magnet or may be made from a (soft) ferromagnetic material. When using a (soft) ferromagnetic material, rather than a permanent magnet, the magnetic element obtains its magnetic properties because of its arrangement in the presence of a magnet, e.g. the magnet of a magnetic stirrer. The magnetic properties then disappear when the magnet is removed. The magnetic element may be made e.g. from neodymium, or other materials with good magnetic properties. In another example, the magnetic element may be made of stainless steel.

The mixing component 30 may comprise a plurality of suitably shaped features for mixing. Such features may include e.g. blades, rings or teeth, which ensure mixing a liquid and gas by creating sufficient turbulence and mixing of the various components as the component is rotated. Mixing may be increased by forced passage through openings or spaces in the disc.

The mixing component may in some examples be freely arranged, i.e. not supported or suspended by any element on the sidewall or the top of the container. The mixing component 30 may comprise a central protrusion which is configured to reduce contact and friction between a bottom of the mixing element with the bottom of the container. The protrusion may be tapered and pointed. When the mixing element starts to rotate, the protrusion maintains contact with the bottom of the container body.

In examples, the mixing component 30 may be a disc or a disc-like component with blades, rings or teeth (or other features shaped for mixing) around an outer perimeter of the disc.

FIG. 2 schematically illustrates a product 100 to produce a foamed sclerosant composition comprising a container as previously discussed.

The product 100 further comprises a sealed package 110 defining an interior space 112 for housing the container 10. The package 110 may include a bottom, and a plurality of sidewalls. A peelable lid 120 may be heat sealed at the top of the package to close off the interior space 112 of the package 110.

The interior space 112 is filled with a gas. Further, the interior space 112 of the sealed package 110 and the foaming space of the container 10 are in fluid communication.

As previously discussed regarding the container 10, the product 100 may be sterilized following any suitable procedure known in the art.

In some examples, the gas in the interior space 112 comprises a percentage of O₂ between 70%-50%, and a percentage of CO₂ between 30%-50%. The gas in the interior space may be composed of less than 10%, specifically less than 5%, more specifically less than 2% and preferably less than 1% nitrogen. Thus, the composition of the gas inside the foaming space of the container 10 and in the interior space 112 of the sealed sterile package 110 may have substantially the same composition, and the mass transfer between these two volumes of gas may not substantially change the gas composition inside the container 10.

As illustrated in FIG. 2 with a broken line, the sealed package may comprise a lid 120 configured to be peeled off by a user of the product, e.g. a medical professional. Once the package 110 is open to the atmosphere, the mass transfer across the gas-permeable barrier 20 of the container 10 will, after some time, result in a change in the composition of the gases inside the container 10. Thus, it may be preferable to use the container 10 to form a foam during the first 15 minutes after opening the package 110, e.g. peeling off the lid 120, and more specifically during the first 5 minutes after opening the package 110.

A suitable time may be determined depending on the barrier. In some examples, a suitable time will be chosen such that the percentage of atmospheric air in the foaming space of the container when preparing the foam is less than 10%, specifically less than 5% and preferably less than 2%.

The sealed package 110 may comprise a gas-impermeable layer, or otherwise be made impermeable to gas. Thus, the sterile package 110 may remain with substantially the same gas composition during a prolonged period of time, e.g. months or even years. In the present example, the gas-impermeable layer may be part of a multi-layer arrangement, further providing rigidity to the package 110 and protecting the container 10, e.g. against sun radiation. More precisely, the illustrated example comprises a multi-layer arrangement comprising an exterior layer of polyethylene, an intermediate layer of ethylene-vinyl alcohol copolymer, and an interior layer of polyethylene. Other examples may comprise different arrangements of the layer(s), such as a single gas-impermeable layer.

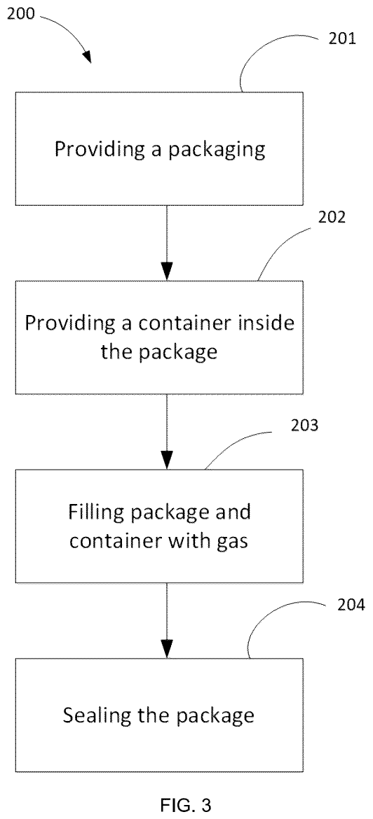

An example of a method 200 for manufacturing a product 100 as previously disclosed is illustrated with reference to FIG. 3.

The method 200 comprises, at block 201, providing a package 110 defining an interior space for housing a container 10. Further, the method 200 comprises, at block 202, providing a container 10 as previously disclosed inside the package 110, wherein the foaming space of the container 10 and the interior space of the package are in fluid communication. Additionally, the method 200 includes, in block 203, filling the package 110 and the container 10 with a physiological gas or mixture of physiological gases. Furthermore, the method 200 comprises, at block 204, sealing the package 110.

As discussed in relation with the package 110 and the container 10, the method 200 may further include a step of sterilization the product 100. Different methods may be used depending on whether the sterilization is performed before or after sealing the package 110.

In some examples, the method 200 further comprises reducing an air pressure around the package below atmospheric pressure before filling the package. Thus, in examples, the air pressure may be brought below 0.1 bar, and more specifically below 0.03 bar. This generates a vacuum condition around the package 110 and container 10, reducing the presence of aerosols and other particles in suspension that may otherwise contaminate the product 100.

In examples, the mixture of physiological gases used to fill the package comprises a percentage of O₂ between 70%-50% and a percentage of CO₂ between 30%-50%. In a specific example, the mixture of physiological gases may comprise a percentage of O₂ of approximately 65% and a percentage of CO₂ of approximately 35%.

In some examples, the step 204 of sealing the package 110 may comprise using a lid 120. The lid 120 may have a multi-layer arrangement. For example, the lid 120 may have a first layer of polyethylene and a second layer of aluminum, but lids with other arrangements and comprising other materials may also be used. Further, in other examples, the step 204 of sealing the package 110 may be performed by bringing together and coupling two ends of the package 110. Thus, sealing the package 110 may be performed applying heat and pressure to the lid 120 against the package 110 or to two ends of the package 110 using a heat sealer machine in a modified atmosphere, e.g. a percentage of O₂ between 70%-50% and a percentage of CO₂ between 30%-50%.

Furthermore, the method 200 may comprise the step of disposing a plurality of products 100 in a parcel configured for product storage and transportation. In addition, the method 200 may also include sterilizing the parcel (and products 100 within) by applying beta or gamma radiation with an ionization radiation between 25 and 55 kilogray (m²s⁻²).

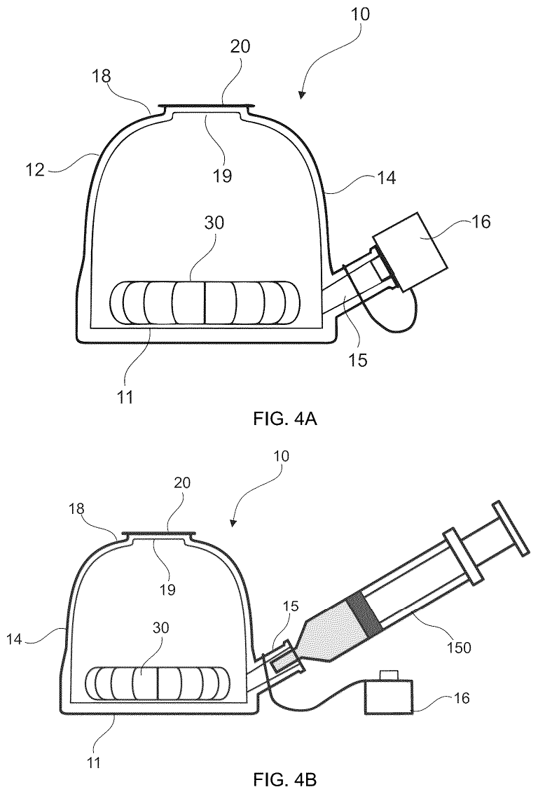

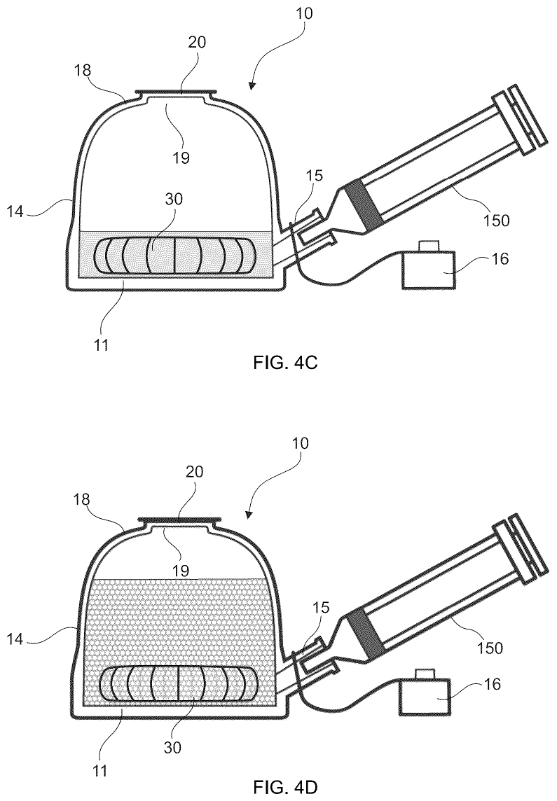

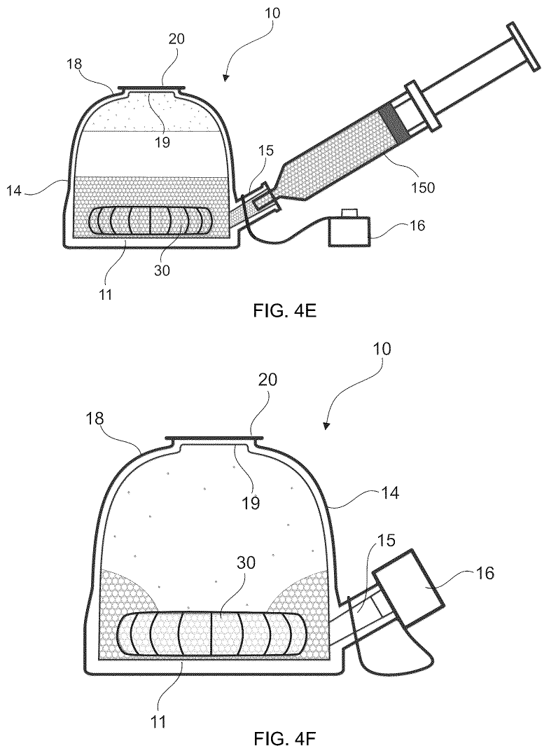

An example of a method for preparing a sclerosant foam with a product 100 according to the present disclosure is illustrated in FIGS. 4A to 4F. Thus, FIGS. 4A to 4F illustrate a set of chronological steps to prepare a sclerosant foam according to the present disclosure. Note that intermediate steps may be included in the foam preparation. Further note that the foam has been schematically illustrated as an area with a pattern of empty circles and that atmospheric air into the container 10 has been illustrated as an area with a pattern of dots.

In FIG. 4A, a product 100 according to the previously shown examples or similar is provided. The product 100 is filled with a mixture of physiological gases and comprises a container 10 in fluid communication with the interior space of the product 100 through a gas-permeable barrier 20. In the illustrated example, the container 10 comprises a stopper 16 to protect the female coupling member 15 against the ingress of contaminants. Once the package 110 is opened and the container 10 is extracted, the stopper 16 may be removed to prepare the female coupling member 15 for the introduction of a tip of a syringe.

At this point, the container 10 will be in fluid communication with the atmospheric air at least through the gas-permeable barrier 20. Further, the container 10 may also be in fluid communication with the atmospheric air through the female coupling member 15, depending on the characteristics of the same. Thus, to limit the diffusion of the mixture of physiological gases from the container 10 into the atmosphere, it is preferable to continue with the steps of the foam preparation in a relatively short period of time, e.g. less than 10 minutes or less than 15 minutes. Depending on the barrier used, and depending on the cross-sectional area of the opening(s), the time that may pass may be shorter or longer than 10 or 15 minutes.

The time that may be allowed to pass may also depend on the desirable composition of the foamed sclerosant composition. For some treatments, a composition of gas inside the container during foaming should be substantially 100% of the original composition, i.e. 100% or very close to it of physiological gases. The composition of the bubbles in the foam will have a mixture of gases that is substantially the same as the composition of the gas inside the container. Foaming should thus occur immediately or almost immediately after opening the package and/or removing the container from the package. For other treatments, a mixture of physiological gases and e.g. 2%, 5%, or a higher percentage of air may be acceptable.

FIG. 4B illustrates how a syringe 150 is connected to the female coupling member 15.

Thus, the method for preparing a sclerosant foam may also comprise providing a syringe 150 filled with a sclerosant liquid composition. In an example, such a syringe 150 may be provided as a pre-filled syringe, possibly as part of the product 100. Alternatively, the syringe 150 may be filled with the sclerosant liquid immediately prior to the preparation of the foam.

In one particular example, the sclerosing composition may comprise a solution of a sclerosing agent, such as e.g. polidocanol, in a liquid, such as water or physiological saline, at a concentration from 2 mg to 30 mg in 1 mL liquid (which corresponds to 0.20-3.0%) (w/v). In another example, the sclerosing composition may comprise a solution of a sclerosing agent, such as polidocanol, in a liquid, such as water or physiological saline, at a concentration from 2 mg to 5 mg in 1 mL liquid (which corresponds to 0.20-0.50% (w/v)). With the devices and methods described herein, it has been found that even at very low concentrations e.g. 0.2% or 0.3% (w/v), still a stable foam may be obtained, contrary to e.g. Tessari's method.

In another particular example, the sclerosing composition may comprise a solution of polidocanol in water or physiological saline at a concentration of 5 mg/mL.

In another particular example, the sclerosing composition may comprise a solution of polidocanol in water or physiological saline at a concentration of 20 mg/mL.

In another particular example, the liquid composition may include Sodium tetradecyl sulfate (STS) at a concentration of 0.2%-3%.

The volume of the syringe may be e.g. 5 cc, 10 cc or 20 cc. The syringe may comprise e.g. 2 ml or more of liquid sclerosant composition.

As discussed, the syringe 150 may be mated with the female coupling member 15 by insertion of the syringe 150. In further examples, the container body 10 may include a one-way valve or a selectively displaceable element to avoid any contamination.

As illustrated in FIG. 4C, once a leak-free fitting between the syringe 150 and the female coupling member 15 is established, the liquid composition inside the syringe 150 may be injected and can enter the foaming space. Depending on the rate of liquid injection, a slight overpressure may be created inside the foaming space, that is, the pressure inside the foaming space may be higher than the external atmospheric pressure. However, this slight overpressure will be quickly equalized due to the presence of the gas-permeable barrier 20. Further, any air or gas inside the syringe 150 may also be injected into the foaming space, although this may be done at any moment prior to the extraction of the foam.

The previously described steps may be carried out in particular after the placement of the container 10 on a magnetic stirrer. This may be a standard magnetic stirrer which may typically be found in many laboratories. In some other examples, the magnetic stirrer may be specifically adapted to the container and may include a receptacle for receiving the container 10. A slight press fit between the container 10 and the receptacle may be provided. The magnetic stirrer may be a stirrer with a magnet driven by a motor, but may also be a magnetic stirrer which is based on induction using magnetic coils which provide for a rotating magnetic field.

As illustrated in FIG. 4D, the magnetic stirrer may then be activated. The mixing component 30 may thus be set in rotation. The mixing component 30, e.g. the disc with magnetic element that was previously described, may rotate on the bottom 11 of the container 10. Depending on the type of foam to be created, depending on the liquid sclerosant composition, and depending on the gas in the foaming space, different revolution speeds and times may be used. In some examples, the magnetic stirrer may include a memory with a number of predefined programs suitable for different foams, such that a user only needs to select a suitable program.

It is noted that it can be advantageous not to remove the syringe 150 during the formation of the foam and maintain it coupled to the container 10.

In particular examples, the magnetic stirrer is first set to a high first speed, which is reached in a short time, for example a few seconds. Such a high first speed may be between 2,000 and 5,000 RPM, and particularly between 2,500 and 4,000 RPM. After as little as 15 seconds, foam may be created.

In order to increase the stability and quality of the foam, the high speed of rotation may be maintained, however. In examples, after a first period, which may be 30-90 seconds, particularly around 60 seconds, the speed of the magnetic stirrer may be reduced to a second speed.

The second speed may be chosen such that the foam that has been created is pushed to the outer perimeter of the foaming space due to centrifugal forces. The second speed may be between 200-2000 RPM in examples. In an example, the second speed may be lower than 1,500 RPM. In an example, the second speed may be between 200 and 1,200 RPM, specifically around 1,000 RPM.

As illustrated in FIG. 4E, once the foam has been stabilized, the foam may be extracted from the container 10. Note that the magnetic stirrer may operate while the foam is extracted. As the gas-permeable barrier 20 allows pressure equalization between an inside of the container 10 and atmospheric pressure, the potential formation of undesirable large bubbles in the sclerosant foam within the collection syringe is avoided.

After extraction of the sclerosant foam, the foam may be used in the treatment of a patient.

The volume of container 10 as disclosed herein may be, for example, 30 ml. Such a container was used in various experiments. With methods according to the examples disclosed herein, approximately 10 ml of stable foam was obtained and easily extracted when introducing 2 ml of liquid sclerosant composition (of any of the hereinbefore mentioned concentrations).

When introducing 4 ml of liquid sclerosant composition, about 20 ml of stable foam was obtained and easily extracted. As illustrated in FIG. 4F, the foam may be extracted in a single step or in multiple steps. For example, a first volume of foam may be extracted and may be used as a first dose in the treatment of a patient. Then, the remaining foam may be kept in the container 10 while the mixing component 30 keeps rotating. Thus, the foam may remain stable for a later use, e.g. a second dose in the treatment of a patient. The stopper 16 may be coupled to the female coupling member 15 to avoid foam contamination and foam spilling between subsequent foam extractions.

Extraction of the foam can cause a lower pressure inside the foaming space, and thus ambient air may be drawn in into the container. Contamination by (micro)particles may nonetheless be avoided. Also, the small quantity of air being drawn into the container does not affect the quality of composition of the already formed foam.

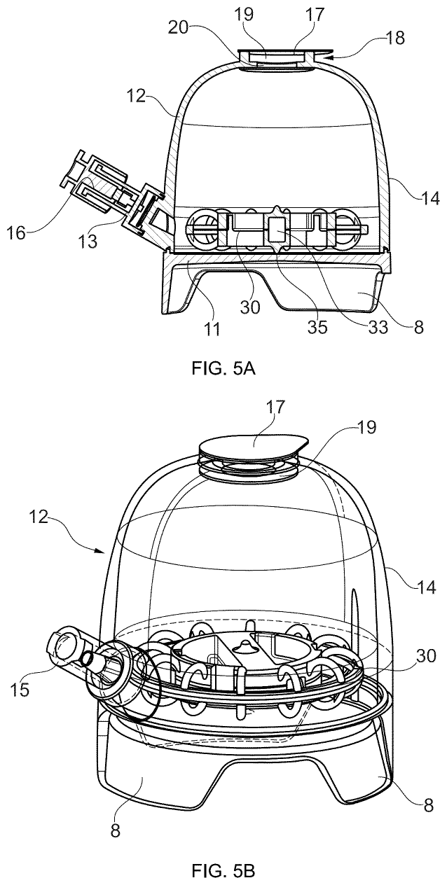

FIGS. 5A and 5B illustrate yet another example of a container according to the present disclosure. The container 12 according to this example has a dome-shape that is similar to the previously illustrated examples, including a sidewall 14 and an orifice 19 arranged near a top 18 of the container body. A foaming space is defined in the interior of the container between the sidewall 14, the top 18 and the bottom 11.

Similarly as in the previous examples, a female coupling member or socket 16 may be arranged in the sidewall. The end of the socket 15 may be arranged relatively close to the bottom 11 such that the socket may be used both for introduction of a liquid sclerosant composition, as well as for extracting the sclerosant from once it has been produced. A stopper 16 may be threadedly coupled to the socket to close the socket.

Also similarly as before, a mixing element 30 is provided inside the foaming space. As previously described, the mixing element may comprise a magnetic or ferromagnetic core 33. A rotating magnetic field may set the mixing element 30 in motion to agitate and mix the liquid and gas to create a foam. The mixing element 30 comprises a centrally arranged pointy protrusion which allows the mixing element to rotate on the bottom 11 of the container body.

The example of FIG. 6 differs from the previously illustrated examples in a few aspects. The socket 15 comprises a valve in this example. At the top 18 of the container body, an orifice 19 is arranged. This orifice 19 may be larger than in the previous example, e.g. 6-8 mm diameter. The orifice 19 is covered at an inside of the container body by a gas-permeable barrier 19. The same orifice 19 is covered at an outside of the container body a gas and fluid tight releasable cover 17. The cover 17 may be an adhesively attached or heat sealed aluminium foil. The foil may be configured to peel off.

In another example, the gas-permeable barrier and the fluid tight cover may both be arranged at an outside of the container body.

The valve in the socket 15 may be a bidirectional valve allowing introduction of liquid and extraction of foam. The valve in this example includes a spring-loaded elastomer disc which closes off the passage to the inside of the container body. The material of the elastomer disc may be silicon for example.

The elastomer disc may be displaced against the action of the spring by the introduction of a syringe. The syringe may push against a stem which in turn displaces the disk allowing fluid passage, e.g. introduction of a liquid sclerosant composition and/or extraction of foam. It should be clear that this is just one example of a valve that may be used.

The base of the container body in this example includes a skirt like extension, including skirt portions 8. The base of the container body, and in this particular case, the skirt like portions 8 may be adapted to a shape of the magnetic stirrer. I.e. the magnetic stirrer and the base of the container may have complementary shapes such that the base fits inside or on top of a support of the magnetic stirrer. The magnetic stirrer may include a recess in which the skirt like portions 8 fit, or the magnetic stirrer may include protrusions between which the skirt like portions 8 fit. Once securely positioned, the magnetic stirrer may be started and the foam may be created.

The container may be delivered in a fluid tight packaging. The packaging and the foaming pace in the container body may comprise gas with the same or substantially the same gas composition. The container body is sealed with the stopper 16 at the female coupling member, and with the fluid tight cover at a top of the container.

In use, a user may introduce a liquid sclerosant composition with a syringe that is coupled to the female coupling member or socket 15. A suitable quantity of liquid sclerosant composition may be introduced into the foaming space. The syringe may remain coupled to the socket, but may also be removed. In such a case, the stopper may close off the socket.

In some examples, the container may include a machine readable identifier (e.g. an RFID tag) which may be registered by the magnetic stirrer. In some examples, the magnetic stirrer may be able to determine the composition of the gas and/or liquid inside the container based on the machine readable identifier. In some examples, the magnetic stirrer may then automatically (pre)select a suitable stirring program.

In other examples, other identifiers or tags may be used, e.g. QR codes, barcode.

The magnetic stirrer may be set in motion as discussed before, optionally using different speeds for different time periods. Before extraction of the foam, the releasable fluid tight cover 17 may be removed. Gas exchange through the gas permeable barrier 20 is thus possible. A slight overpressure that may be created upon introduction of the liquid sclerosant composition can thus be released. Upon extraction of the foam, a lower pressure may be created inside the foaming space, and thus some ambient air may be introduced through the gas permeable barrier.

The process of filling the container of this example may also be slightly different than the process described earlier. In this particular case, first the container body may be assembled, and the female coupling member may be closed off with the stopper. The combination of stopper and valve in the female coupling member ensure that the female coupling member is sealed off.

The orifice at the top of the container body may be covered (at the inside or at the outside of the container, depending on the embodiment) with the gas permeable barrier. Filling of the container with a suitable gas composition may take place as before whereas the gas passes through the gas permeable barrier. Since in this example, the only gas exchange occurs at the gas permeable barrier, the orifice may be larger than in the previous examples to ensure that filling of the container can occur in a reasonable time frame. After the container has been filed, the fluid tight releasable cover may seal off the container.

After this process has been completed, the container with the desired gas composition may be introduced into the packaging as described before. The side of the packaging may then be filled with the same gas composition and the packaging is then closed.

Note that the technical features disclosed in relation to the containers, products, kits, systems and methods may be interchangeably used. For example, a technical feature disclosed in relation to a container may be integrated into a method to manufacture a product and vice versa.

Some aspects of the present disclosure are set out in the following numbered clauses:

Clause 1. A container for the production of a foamed sclerosant composition, the container comprising:
a container body for foaming comprising one or more sidewalls extending between a top and a bottom of the container body, a foaming space being defined in an interior of the container body,
the foaming space containing a mixing component configured for rotating and comprising a magnetic part and a shape suitable for mixing, and configured to be operatively coupled with a magnetic stirrer, wherein
the container comprises a coupling member for mating with a tip of a syringe, the coupling member being located at or near the bottom surface of the container body such that the syringe can be used for extracting the foamed sclerosant composition, and
the container comprises at least one opening which is covered by a gas-permeable barrier.

Clause 2. The container according to clause 1, wherein the gas-permeable barrier comprises a non-woven fabric.

Clause 3. The container according to any of clauses 1 and 2, wherein the gas-permeable barrier comprises polypropylene, polyethylene or a combination of the same.

Clause 4. The container according to any of clauses 1-3, wherein the coupling member comprises a barrier, optionally a gas-permeable barrier.

Clause 5. The container according to any of clauses 1-4, wherein the opening is arranged at or near a top of the container body.

Clause 6. The container according to any of clauses 1-5, wherein the opening is further covered by a fluid-tight cover.

Clause 7. The container according to clause 6, wherein the gas-permeable barrier is arranged at an inside of the container, and the fluid-tight cover is arranged at an outside of the container.

Clause 8. The container according to clause 6 or 7, wherein the fluid tight cover is an aluminium foil.

Clause 9. The container according to any of clauses 6-8, wherein the fluid-tight cover is heat sealed to the container.

Clause 10. The container according to any of clauses 1-9, wherein the container comprises a sterile physiological gas or mixture of physiological gases, and optionally is a mixture of O₂, and CO₂.

Clause 11. The container according to clause 10, wherein a percentage of O₂ is between 70%-50% and a percentage of CO₂ is between 30%-50%.

Clause 12. The container according to clause 11, wherein a percentage of nitrogen is less than 5%, specifically less than 2%.

Clause 13. A product to produce a foamed sclerosant composition comprising a container according to any of clauses 1-12, the product further comprising:
a sealed package defining an interior space for housing the container, wherein the interior space is filled with a gas, and the interior space of the sealed package and the foaming space are in fluid communication.

Clause 14. The product according to clause 13, wherein the sealed package comprises a lid configured to be peeled off.

Clause 15. The product according to clause 13 or 14, wherein the sealed package comprises a gas impermeable layer.

Clause 16. The product according to clause 15, wherein the gas impermeable layer comprises ethylene-vinyl alcohol copolymer.

Clause 17. The product according to any of clauses 13-16, wherein the sealed package comprises a multi-layer arrangement comprising polyethylene.

Clause 18. A method for manufacturing a product according to any of clauses 13-17, wherein the method comprises:
providing a package defining an interior space for housing the container,
providing a container according to any of clauses 1-5 inside the package, wherein the interior space of the package and the foaming space of the container are in fluid communication through the gas-permeable barrier,
filling the package and the container with a physiological gas or mixture of physiological gases, and
sealing the package.

Clause 19. The method according to clause 18, wherein the method further comprises:
reducing an air pressure around the package below atmospheric pressure before filling the package.

Clause 20. A method for preparing and extracting a foamed sclerosant composition, comprising:
providing a product according to any of clauses 13-17;
opening the package;
removing the container from the package;
mating a syringe with the coupling element of the container and injecting a liquid sclerosant composition into the foaming space of the container;
positioning the container on a magnetic stirrer;
activating the magnetic stirrer to rotate the mixing component and create a foamed sclerosant composition; and
extracting the obtained foamed sclerosant composition, wherein
the foamed sclerosant composition is created within 15 minutes or less from opening the package, specifically within 10 minutes or less form opening the package.

Although only a number of examples have been disclosed herein, other alternatives, modifications, uses and/or equivalents thereof are possible. Furthermore, all possible combinations of the described examples are also covered.

1. A product to produce a foamed sclerosant composition, the product comprising:
a container for the production of a foamed sclerosant composition; and
a sealed package having an interior space housing the container, wherein the interior space is filled with a gas,
the container including:
a container body comprising one or more sidewalls extending between a top and a bottom of the container body, a foaming space being defined in an interior of the container body,
the foaming space containing a mixing component configured for rotating and comprising a magnetic part and a shape suitable for mixing, and configured to be operatively coupled with a magnetic stirrer, wherein the container comprises a coupling member for mating with a tip of a syringe, and the container comprises an opening which is covered by a first gas-permeable barrier and wherein the interior space of the sealed package and the foaming space in the interior of the container body are filled with substantially the same gas.

2. The product according to claim 1, wherein the first gas-permeable barrier comprises a non-woven fabric.

3. The product according to claim 1, wherein the first gas-permeable barrier comprises polypropylene, polyethylene or a combination of the same.

4. The product according to claim 1, wherein the coupling member of the container body includes an opening covered by a second gas-permeable barrier.

5. The product according to claim 1, wherein the opening of the container is arranged at or near the top of the container body.

6. The product according to claim 5, wherein the first gas-permeable barrier covered by a fluid tight cover.

7. The product according to claim 1, wherein the gas comprises a sterile physiological gas or mixture of physiological gases.

8. The product according to claim 1, wherein the gas comprises a mixture of O₂ and CO₂.

9. The product according to claim 7, wherein the gas comprises less than 10% of nitrogen.

10. The product according to claim 8, wherein the gas comprises 50% to 70% of O₂i and 30% to 50% of CO₂.

11. The product according to claim 1, wherein the interior space of the sealed package and the foaming space of the container are in fluid communication through the first gas permeable barrier.

12. The product according to claim 1, wherein the sealed package comprises a lid configured to be peeled off.

13. The product according to claim 1, wherein the sealed package comprises a gas impermeable material.

14. The product according to claim 13, wherein the gas impermeable layer comprises ethylene-vinyl alcohol copolymer.

15. A method for manufacturing a product comprising:
providing a package defining an interior space for housing the container,
providing a container inside the package, the container comprising a container body for foaming having one or more sidewalls extending between a top and a bottom of the container body, a foaming space being defined in an interior of the container body, and the foaming space containing a mixing component configured for rotating and comprising a magnetic part and a shape suitable for mixing, and configured to be operatively coupled with a magnetic stirrer, wherein
the container comprises a coupling member for mating with a tip of a syringe, and further comprises an opening with a gas-permeable barrier, and the method further comprising:
filling the package and the container with a physiological gas or mixture of physiological gases, and
sealing the package.

16. The method of claim 15, wherein the interior space of the package and the foaming space of the container are in fluid communication through the gas-permeable barrier during filling.

17. The method of claim 15, wherein the method further comprises:
reducing an air pressure around the package below atmospheric pressure before filling the package.

18. The method of claim 15, the method further comprising sealing off the opening of the container body with a fluid tight releasable cover after filling the container with the physiological gas or mixture of physiological gases.

19. The method of claim 18, wherein the sealing off the opening of the container body is carried out before filling the package with the physiological gas or mixture of physiological gases.

20. A method for preparing and extracting a foamed sclerosant composition, comprising:
providing a product according to claim 1;
opening the sealed package;
removing the container from the opened sealed package;
mating a syringe with the coupling member of the container and injecting a liquid sclerosant composition into the foaming space of the container;
positioning the container on the magnetic stirrer;
activating the magnetic stirrer to rotate the mixing component and create a foamed sclerosant composition; and
extracting the obtained foamed sclerosant composition, wherein
the foamed sclerosant composition is created within 15 minutes or less from a moment of opening the sealed package.
